(19) United States
(12) Patent Application Publication
(10) Pub. No.: US 2016/0192826 A1
(43) Pub. Date: Jul. 7, 2016

(54) ENDOSCOPE DISTAL END CAP ATTACHING DEVICE, ENDOSCOPE DISTAL END CAP AND ENDOSCOPE APPARATUS

(71) Applicant: OLYMPUS CORPORATION, Tokyo (JP)

(72) Inventors: Mayumi SHIMAZAKI, Hino-shi (JP); Tae MITSUYA, Machida-shi (JP)

(73) Assignee: OLYMPUS CORPORATION, Tokyo (JP)

(21) Appl. No.: 15/071,638

(22) Filed: Mar. 16, 2016

Related U.S. Application Data
(63) Continuation of application No. PCT/JP2014/078788, filed on Oct. 29, 2014.

(30) Foreign Application Priority Data
Nov. 15, 2013 (JP) 2013-237431

(51) Int. Cl. A61B 1/00; A61B 1/018

(52) U.S. Cl. CPC A61B 1/00137; A61B 1/018

(57) ABSTRACT

An endoscope distal end cap attaching device attaches an endoscope distal end cap to an inserting distal end portion of an endoscope inserting section. The endoscope distal end cap attaching device includes an attaching inserting section that is inserted to an opening portion, an abutment section having one or more abutment distal end surface portions that abut only on at least a part of a non-functional region portion, a base section which has a base distal end surface portion, the base distal end surface portion being disposed away from an abutment distal end surface portion of the abutment section, and a positioning section which positions the endoscope distal end cap and the endoscope distal end cap attaching device.

### CROSS-REFERENCE TO RELATED APPLICATIONS

This application is a Continuation application of PCT Application No. PCT/JP2014/078788, filed Oct. 29, 2014 and based upon and claiming the benefit of priority from prior Japanese Patent Application No. 2013-237431, filed Nov. 15, 2013, the entire contents of all of which are incorporated herein by reference.

### BACKGROUND OF THE INVENTION

1. Field of the Invention

The present invention relates to an endoscope distal end cap attaching device, an endoscope distal end cap and an endoscope apparatus.

2. Description of the Related Art

When an inserting section of an endoscope is inserted to, for example, a stomach, a mucous membrane of the stomach might adhere to an observation window portion disposed in a distal end portion of the inserting section. Consequently, a desirable field of view might not be acquired. To eliminate such a trouble, an endoscope distal end cap (hereinafter referred to as the cap) that prevents the mucous membrane from adhering to the observation window portion is attached to the distal end portion of the inserting section.

Such caps are disclosed in, for example, Jpn. Pat. Appln. KOKAI Publication No. 2003-290132, Jpn. Pat. Appln. KOKAI Publication No. 2003-116772 and Jpn. Pat. Appln. KOKAI Publication No. 2003-116773. When the cap is attached to the distal end portion, an attaching operation of the cap to the distal end portion takes time and labor, because the cap is soft and easy to be deformed. Additionally, an operation of appropriately positioning the cap in a periaxial direction of the inserting section and a periaxial direction of the cap to attach the cap to the distal end portion takes time and labor.

In consequence, the cap is attached to the distal end portion by using such an endoscope distal end cap attaching device (hereinafter referred to as the attaching device) as disclosed in, for example, Jpn. Pat. Appln. KOKAI Publication No. 2003-116772 and Jpn. Pat. Appln. KOKAI Publication No. 2003-116773.

### BRIEF SUMMARY OF THE INVENTION

An aspect of an endoscope distal end cap attaching device attaches an endoscope distal end cap to an inserting distal end portion of an endoscope inserting section. The inserting distal end portion has a distal end surface including a functional region portion in which a window portion for observation or illumination of a subject is disposed, one or more opening portions, and a non-functional region portion that is a region excluding the functional region portion and the opening portion. The endoscope distal end cap attaching device includes an attaching inserting section that is inserted to the opening portion, when the endoscope distal end cap attaching device attaches the endoscope distal end cap to the inserting distal end portion; an abutment section having one or more abutment distal end surface portions that abut only on at least a part of the non-functional region portion except the functional region portion, when the attaching inserting section is inserted to the opening portion; a base section which has a base distal end surface portion on which the abutment section is disposed, the base distal end surface portion being disposed away from the abutment distal end surface portion of the abutment section in an axial direction of the endoscope distal end cap; and a positioning section which positions the endoscope distal end cap and the endoscope distal end cap attaching device in a periaxial direction of the endoscope inserting section by the abutment of a cap distal end surface of the endoscope distal end cap, when the abutment distal end surface portion abuts on the non-functional region portion, the positioning section being disposed around the base section.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1A is a schematic perspective view of an endoscope apparatus according to a first embodiment of the present invention;

FIG. 1B is a perspective view of an endoscope distal end cap attached to a distal end hard portion;

FIG. 1C is a perspective view of the endoscope distal end cap shown in FIG. 1B;

FIG. 1D is a view showing an observation view field when the endoscope distal end cap shown in FIG. 1C is attached to the distal end hard portion;

FIG. 1E is a perspective view of a cylindrical endoscope distal end cap different from the endoscope distal end cap of the present embodiment;

FIG. 1F is a view showing an observation view field when the endoscope distal end cap shown in FIG. 1E is attached to the distal end hard portion;

FIG. 2A is a front view of the distal end hard portion to which the endoscope distal end cap is attached;

FIG. 2B is a perspective view of the distal end hard portion, the endoscope distal end cap and an endoscope distal end cap attaching device;

FIG. 2C is a perspective view of a state where the endoscope distal end cap is attached to the distal end hard portion by using the endoscope distal end cap attaching device;

FIG. 2D is a front view of the endoscope distal end cap attaching device;

FIG. 3A is a view showing a relation between a length and a diameter in each of the endoscope distal end cap and the endoscope distal end cap attaching device;

FIG. 3B is a view showing a state where an inserting distal end portion of an attaching inserting section passes through the endoscope distal end cap, projects from a proximal end portion of the endoscope distal end cap and is exposed from the proximal end portion, when the endoscope distal end cap is attached to the endoscope distal end cap attaching device;

FIG. 3C is a view showing that the attaching inserting section is shorter than a hard member;

FIG. 4A is a view showing that an exposed surface is formed as an outward flange portion;

FIG. 4B is a view showing that the inserting distal end portion of the attaching inserting section has a chamfered portion;

FIG. 4C is a view showing that the attaching inserting section is formed to be tapered from an inserting proximal end portion toward an inserting distal end portion in an axial direction of the attaching inserting section;

FIG. 5A is a view showing that the attaching inserting section is inserted to a discharge opening portion;

FIG. 5B is a view showing that the attaching inserting sections are inserted to treatment device opening portions;

FIG. 5C is a view showing that the attaching inserting sections are inserted into the treatment instrument opening portion and the discharge opening portion;

FIG. 6A is a view showing a state where a part of a circular outer peripheral surface of an abutment section is disposed on the same plane as that of a part of an outer peripheral surface of a base section in an axial direction of the attaching device, and connected to the part of the outer peripheral surface of the base section;

FIG. 6B is a side view of FIG. 6A;

FIG. 7A is a view showing that the abutment sections are disposed; and

FIG. 7B is a view showing that the abutment sections are disposed.

### DETAILED DESCRIPTION OF THE INVENTION

Hereinafter, embodiments of the present invention will be described in detail with reference to the drawings. It is to be noted that parts of the drawings omit parts of members for clarification of the drawing.

### First Embodiment

### Configuration

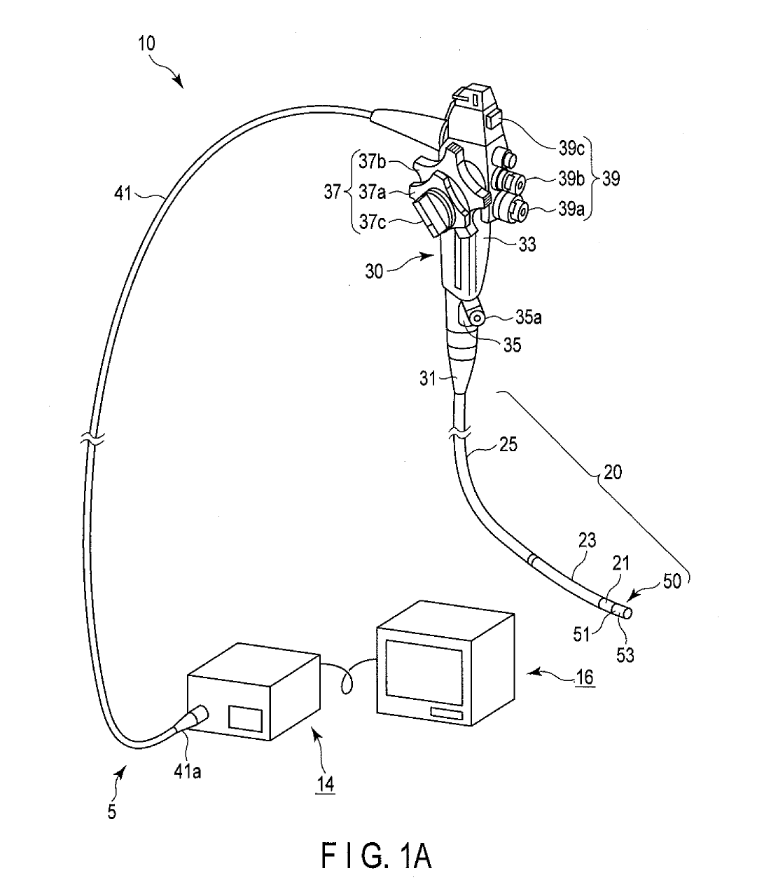

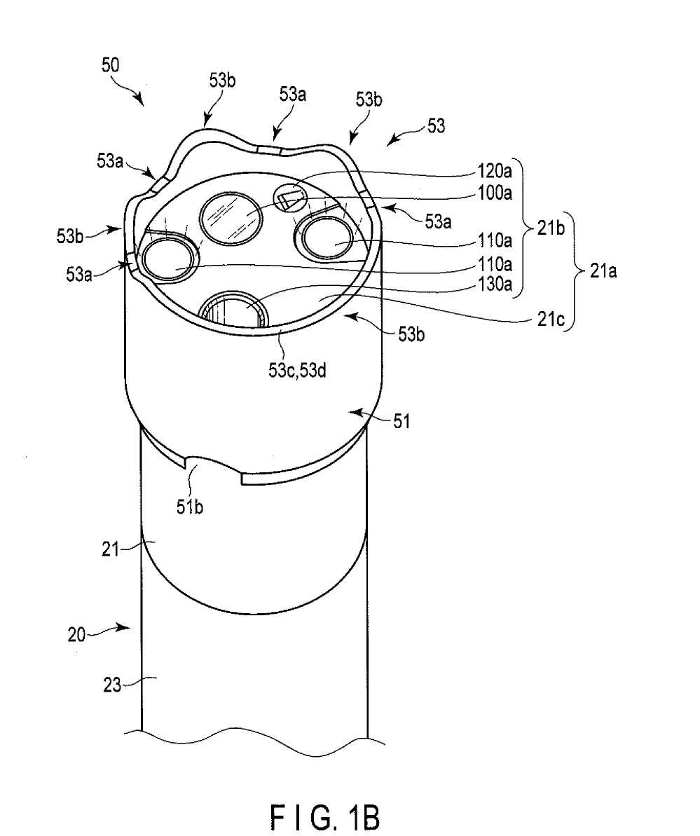

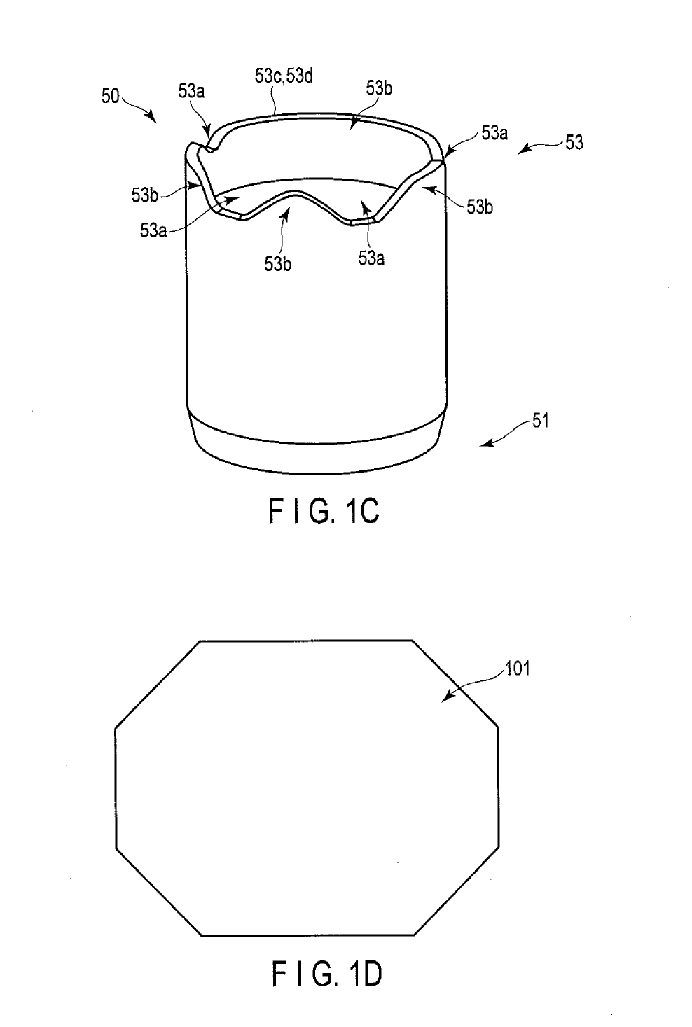

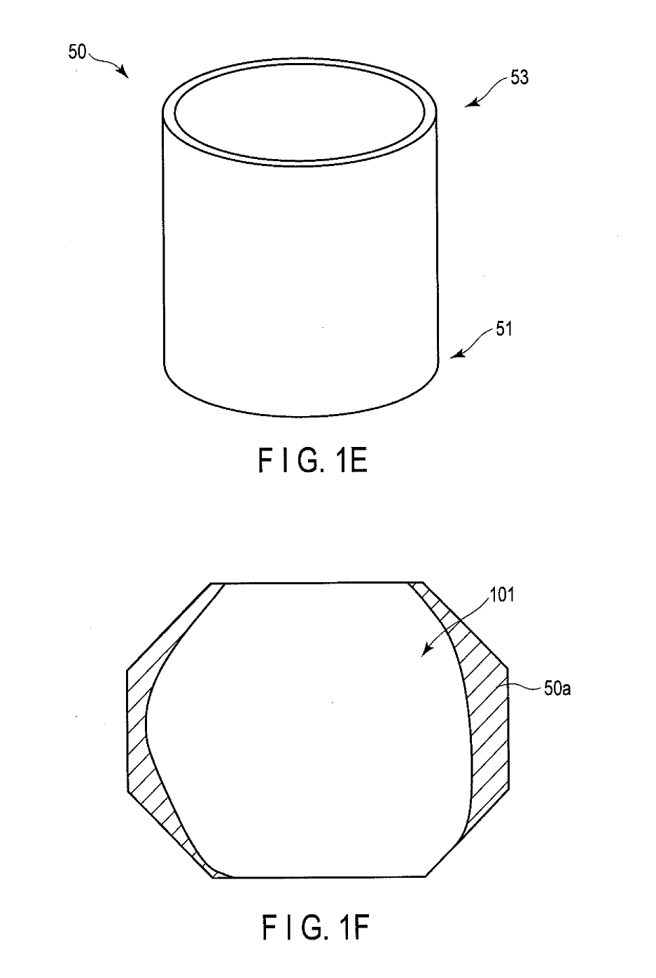

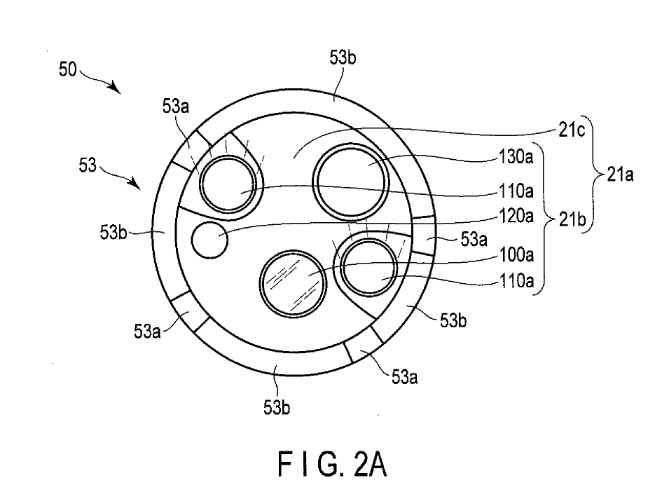

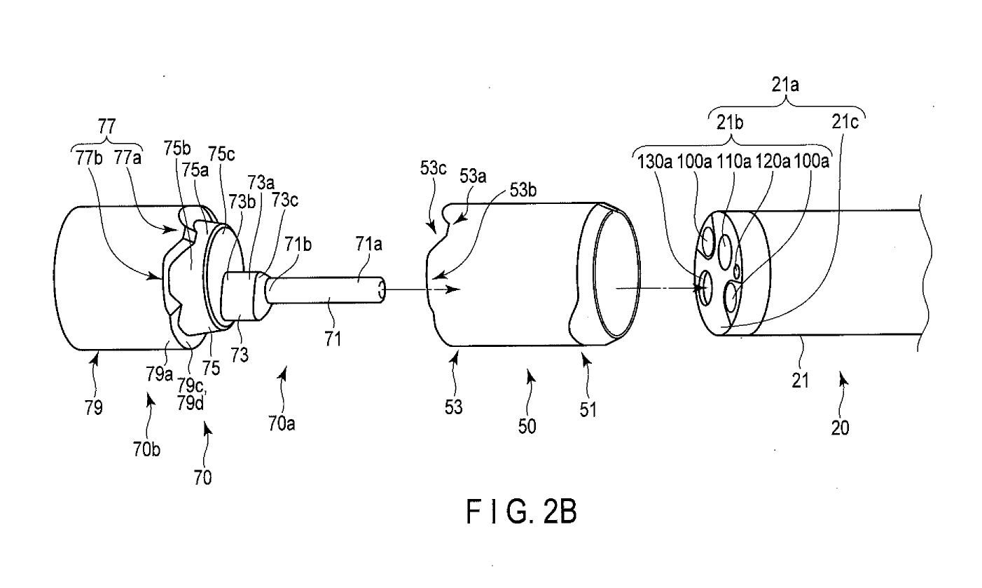

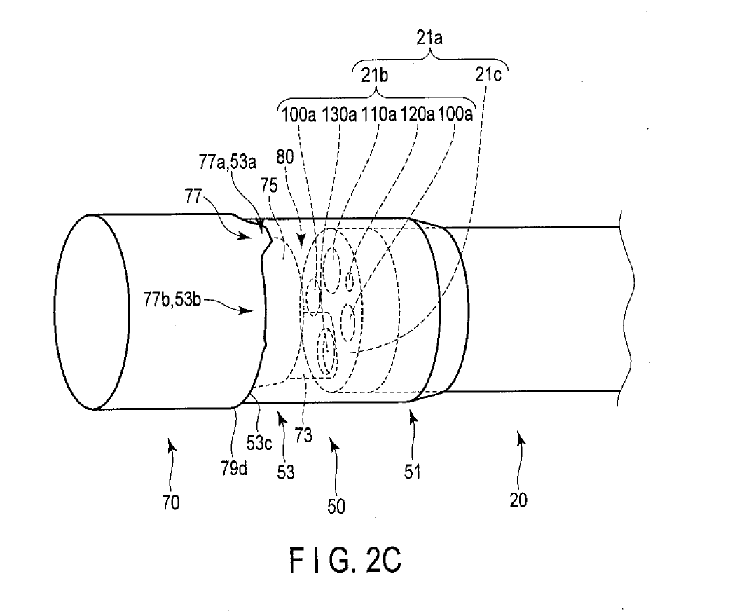

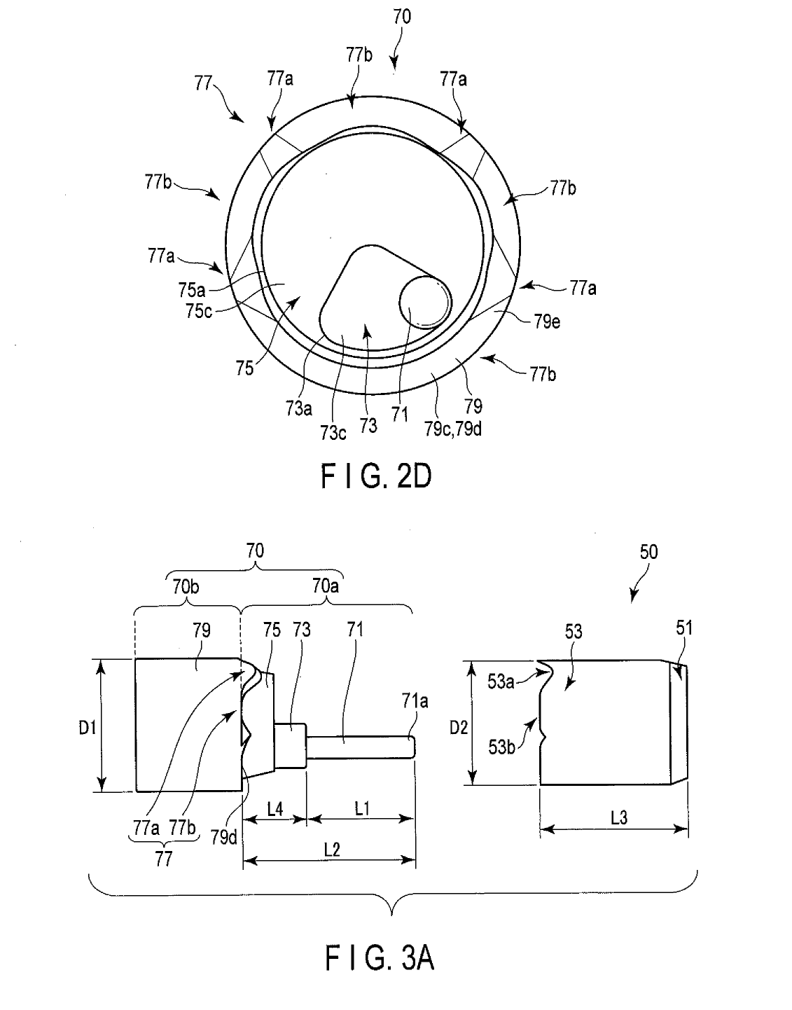

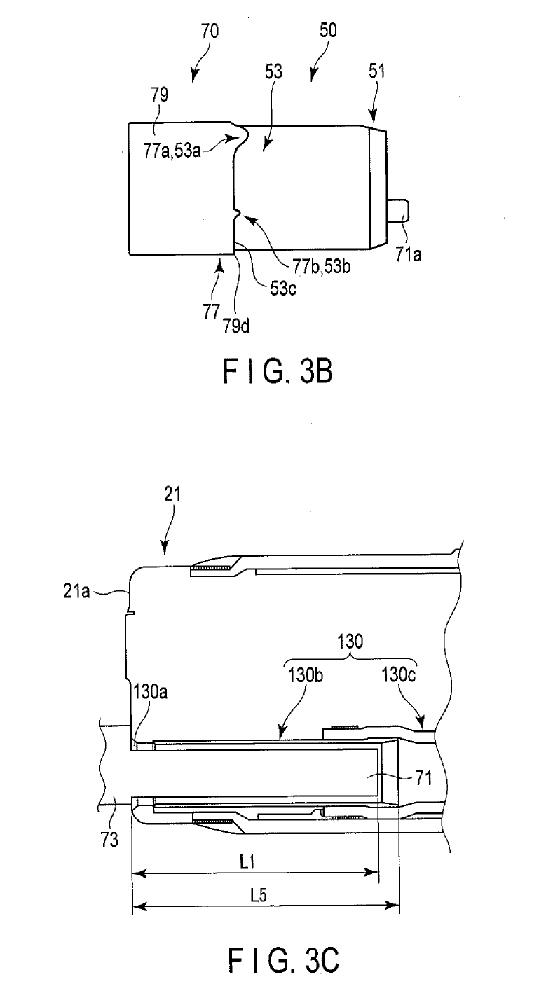

A first embodiment will be described with reference to FIG. 1A, FIG. 1B, FIG. 1C, FIG. 1D, FIG. 1E, FIG. 1F, FIG. 2A, FIG. 2B, FIG. 2C, FIG. 2D, FIG. 3A, FIG. 3B and FIG. 3C.

[Endoscope Apparatus 5]

As shown in FIG. 1, an endoscope apparatus 5 has an endoscope 10 having an unshown imaging unit, a control device 14 that controls the endoscope 10, and a display section 16 connected to the control device 14. The control device 14 has an unshown image processing section that processes an image imaged by the imaging unit. The display section 16 displays the image imaged by the endoscope 10 and processed by the image processing section.

[Endoscope 10]

The endoscope 10 shown in FIG. 1A is, for example, a direct viewing type of endoscope.

As shown in FIG. 1A, the endoscope 10 has an elongated inserting section 20 to be inserted into, for example, a patient's lumen, and an operating section 30 coupled with a proximal end portion of the inserting section 20 to operate the endoscope 10.

[Operating Section 20]

As shown in FIG. 1A, the inserting section 20 has a distal end hard portion 21, a bending portion 23, and a flexible tube portion 25 from a distal end portion side of the inserting section 20 toward the proximal end portion side of the inserting section 20. A proximal end portion of the distal end hard portion 21 is coupled with a distal end portion of the bending portion 23, and a proximal end portion of the bending portion 23 is coupled with a distal end portion of the flexible tube portion 25.

The distal end hard portion 21 is the distal end portion of the inserting section 20, and is hard and does not bend. A configuration of the distal end hard portion 21 will be described later. The distal end hard portion 21 functions as an inserting distal end portion of the inserting section 20.

The bending portion 23 is bent in a desirable direction such as an upward, downward, right or left direction by an operation of a bending operation portion 37 which will be described later. When the bending portion 23 is bent, a position and an orientation of the distal end hard portion 21 change. Further, an observation object is illuminated with unshown illumination light, and the observation object is captured in an observation view field. This observation object is, for example, an affected area, a lesioned area or the like in the subject (e.g., a body cavity).

The flexible tube portion 25 is a tubular member extended from a main body portion 31 which will be described later in the operating section 30, and has a desirable flexibility so that it can be bent by an external force.

[Operating Section 30]

As shown in FIG. 1A, the operating section 30 has the main body portion 31 from which the flexible tube portion 25 is extended, a grasping section 33 that is coupled with a proximal end portion of the main body portion 31 and is grasped by an operator who operates the endoscope 10, and a universal cord 41 connected to the grasping section 33.

[Grasping Section 33]

As shown in FIG. 1A, the grasping section 33 has a treatment instrument inserting portion 35, the bending operation portion 37 that bends and operates the bending portion 23, and a switch portion 39. The treatment instrument inserting portion 35 is disposed in a distal end portion of the grasping section 33, and the bending operation portion 37 and the switch portion 39 are disposed in a proximal end portion of the grasping section 33.

[Treatment Instrument Inserting Portion 35]

As shown in FIG. 1A, the treatment instrument inserting portion 35 is branched to the grasping section 33. Consequently, a central axis direction of the treatment instrument inserting portion 35 slants in a central axis direction of the grasping section 33.

As shown in FIG. 1A, the treatment instrument inserting portion 35 has a treatment instrument insertion port 35a which is disposed at an end portion of the treatment instrument inserting portion 35 and to which an unshown treatment instrument is inserted the endoscope 10.

The treatment instrument insertion port 35a is coupled with a proximal end portion of an unshown treatment instrument inserting channel. The treatment instrument inserting channel is disposed inside the inserting section 20, and disposed from the flexible tube portion 25 to the distal end hard portion 21 via the bending portion 23. A distal end portion of the treatment instrument inserting channel communicates with a treatment instrument opening portion 130a (see FIG. 1B) disposed in the distal end hard portion 21. The treatment instrument insertion port 35a is an insertion port to which the treatment instrument is inserted the treatment instrument inserting channel.

[Bending operation portion 37]

As shown in FIG. 1A, the bending operation portion 37 has a right/left curving operation knob 37a that bends and operates the bending portion 23 to right and left, an upward/downward curving operation knob 37b that bends and operates the bending portion 23 upward and downward, and a bend fixing knob such as a fixing knob 37c that fixes a position of the bending portion 23 which is bent.

[Switch Portion 39]

As shown in FIG. 1A, the switch portion 39 has a suction switch 39a, a gas sending/water sending switch 39b, and various switches 39c for endoscope photography. The suction switch 39a, the gas sending/water sending switch 39b and the various switches 39c are operated by an operator's hand when the grasping section 33 is grasped by the operator.

The suction switch 39a is operated, when the endoscope 10 sucks mucus, fluid or the like from the treatment instrument opening portion 130a that also serves as a suction opening portion via the treatment instrument inserting channel that also serves as a suction channel.

For the purpose of acquiring an observation view field 101 (see FIG. 1D) of an observation unit in the distal end hard portion 21, the gas sending/water sending switch 39b is operated when the fluid is sent from an unshown gas sending tube and an unshown gas sending/water sending tube, and when the fluid is sent from the water sending tube and the gas sending/water sending tube. The fluid includes water or a gas.

The gas sending tube, the water sending tube and the gas sending/water sending tube are disposed from the inserting section 20 via the main body portion 31 and the grasping section 33 up to the universal cord 41 in the endoscope 10.

[Universal Cord 41]

As shown in FIG. 1A, the universal cord 41 is extended from a side surface of the grasping section 33. The universal cord 41 has a connecting connector 41a that is detachable from and attachable to the control device 14.

[Endoscope Distal End Cap (Hereinafter Referred to as a Cap 50)]

As shown in FIG. 1A, FIG. 1B and FIG. 1C, the endoscope 10 further has the cap 50 that is attachable to and detachable from the distal end hard portion 21 functioning as the distal end portion of the inserting section 20. The cap 50 has a tubular shape, e.g., a substantially cylindrical shape. The cap 50 is made of a soft material such as a resin.

As shown in FIG. 1A and FIG. 1B, the cap 50 has a proximal end portion 51 detachably attached to the distal end portion of the distal end hard portion 21, and a distal end portion 53 disposed forward to a distal end surface 21a of the distal end hard portion 21 when the proximal end portion 51 is attached to the distal end portion of the distal end hard portion 21. That is, the cap 50 is detachably attached to the distal end hard portion 21 so that in an axial direction of the cap 50, the proximal end portion 51 that is a part of the cap 50 is attached to the distal end hard portion 21 and the distal end portion 53 that is a residual part of the cap 50 projects forward from the distal end surface 21a of the distal end hard portion 21. Further, the proximal end portion 51 functions as an attaching portion that attaches the cap 50 to the distal end hard portion 21.

As shown in FIG. 1B, the distal end surface 21a has, for example, an observation window portion 100a of the observation unit to observe the subject, and an illumination window portion 110a of an illumination unit that illuminates the subject with the illumination light. The distal end surface 21a further has a gas sending/water sending nozzle 120a that sends the gas and water toward the observation window portion 100a and the treatment instrument opening portion 130a from which the treatment instrument projects.

As shown in FIG. 1B and FIG. 2A, the treatment instrument opening portion 130a is disposed at a position different from that on a central axis of the distal end surface 21a. For example, the treatment instrument opening portion 130a is disposed eccentrically from the central axis of the distal end surface 21a toward an edge portion side of the distal end surface 21a. That is, the treatment instrument opening portion 130a is eccentric to the central axis of the distal end surface 21a.

As shown in FIG. 1B and FIG. 2A, the distal end surface 21a has a functional region portion 21b, and a non-functional region portion 21c that is a region excluding the functional region portion 21b. The functional region portion 21b has the observation window portion 100a, the illumination window portion 110a, the gas sending/water sending nozzle 120a and an opening portion such as the treatment instrument opening portion 130a. The opening portion is eccentric to the central axis of the distal end surface 21a. The non-functional region portion 21c indicates a region portion disposed in the distal end surface 21a other than the functional region portion 21b disposed in the distal end surface 21a. The non-functional region portion 21c has, for example, a planar shape. The functional region portion 21b is a first region portion in which functioning members are disposed. The non-functional region portion 21c is a second region portion in which the functioning members are not disposed.

[Concave Portion 53a of Cap 50]

A projecting amount of the distal end portion 53 projecting from the distal end surface 21a, i.e., a projecting height of the cap 50 is set to, for example, an optimum distance in close observation. Specifically, the distal end hard portion 21 has the observation unit and the illumination unit. As shown in FIG. 1D, the observation view field 101 of the observation unit has a non-circular shape such as a substantially octagonal shape. There will be described a case where the cylindrical cap 50, in which concave portions 53a and convex portions 53b are not disposed in the distal end portion 53 as shown in FIG. 1E, is attached to the distal end hard portion 21. The cap 50 shown in FIG. 1E is different from the cap 50 of the present embodiment. In this case, as shown in FIG. 1F, a part 50a of a distal end of the cap 50 enters into both side portions of the observation view field 101, and the part 50a might obstruct the observation view field 101. Although not shown, the part 50a of the distal end of the cap 50 might enter into both side portions of an illumination range and the part 50a might obstruct the illumination range.

To eliminate such a trouble, as shown in FIG. 1B and FIG. 1D, the cap 50 is formed so that the distal end portion 53 projecting from the distal end surface 21a does not obstruct the observation view field 101 of the observation unit and the illumination range of the illumination unit when the cap 50 is attached to the distal end hard portion 21. In detail, as shown in FIG. 1B, FIG. 1C and FIG. 1D, the part 50a of the distal end of the cap 50 is made concave so that the cap 50 does not enter into the observation view field 101 and the illumination range. Therefore, as shown in FIG. 1B, FIG. 1C and FIG. 2A, for example, the cap 50 has the concave portions 53a disposed in the distal end portion 53. The concave portions 53a are made concave from the distal end portion 53 toward the proximal end portion 51 in the axial direction of the cap 50. The concave portion 53a may be adjacent to the observation unit in a radial direction of the cap 50 so that the concave portion 53a overlaps with the observation view field 101 of the observation unit which is disposed in the distal end hard portion 21. The concave portion 53a may be adjacent to the illumination unit in the radial direction of the cap 50 so that the concave portion 53a overlaps with the illumination range of the illumination unit disposed in the distal end hard portion 21. At least one of the above two overlapped states may be made. Consequently, the concave portions 53a are disposed so that, for example, the concave portions 53a are adjacent to the observation window portion 100a and the illumination window portion 110a. It is to be noted that at least parts of the concave portions 53a may be disposed so that at least the parts of the concave portions 53a are adjacent to the observation window portion 100a and the illumination window portion 110a in accordance with a shape of a mask in an endoscope observation image, in accordance with a region where the illumination light may be obstructed, in accordance with a region where the illumination light does not have to be obstructed, or in accordance with a breadth of the observation view field 101 or the illumination light. The concave portions 53a are disposed in the part 50a of the cap 50 in a periaxial direction of the cap 50. The concave portions 53a are arranged in the periaxial direction of the cap 50.

[Convex Portion 53b of Cap 50]

Further, as shown in FIG. 1B, FIG. 1C and FIG. 2A, the convex portions 53b are disposed adjacent to the concave portions 53a in the periaxial direction of the cap 50. The concave portions 53a and the convex portions 53b are alternately arranged in the periaxial direction of the cap 50. The concave portions 53a and the convex portions 53b are disposed in an edge portion of the distal end of the cap 50. The concave portions 53a and the convex portions 53b function as the distal end of the cap 50.

Shapes, heights and the like of the concave portions 53a and the convex portions 53b are desirably set in accordance with the shape of the mask in the endoscope observation image, in accordance with the region where the illumination light may be obstructed, in accordance with the region where the illumination light does not have to be obstructed, or in accordance with the breadth of the observation view field 101 or the illumination light.

[Distal end surface 53c of Cap 50]

As shown in FIG. 1B and FIG. 1C, in the distal end portion 53 of the cap 50 having the concave portions 53a and the convex portions 53b in this manner, a distal end surface 53c of the distal end portion 53 is formed in, for example, a wave shape in the periaxial direction of the cap 50. Consequently, the projecting amount of the distal end of the cap 50 partially changes. In the periaxial direction of the cap 50, a ridgeline portion 53d of the distal end surface 53c is smoothened. That is, the concave portions 53a are smoothly continuous with the convex portions 53b without forming any stepped portions in the periaxial direction of the cap 50.

[Index Portion 51b]

As shown in FIG. 1B, the proximal end portion 51 has an index portion 51b disposed to position the distal end hard portion 21 and the cap 50 with each other in a periaxial direction of the distal end hard portion 21 and the periaxial direction of the cap 50 so that the concave portions 53a are disposed adjacent to the observation unit and the illumination unit as described above. When the index portion 51b is disposed in a desirable portion to be adjusted in accordance with, for example, a position or a width of the treatment instrument opening portion 130a in the periaxial direction of the distal end hard portion 21, the concave portions 53a are disposed adjacent to the observation unit and the illumination unit as described above. The index portion 51b is formed by disposing, for example, a cutout portion in a part of the proximal end portion 51.

[Endoscope Distal End Cap Attaching Device (Hereinafter Referred to as an Attaching Device 70)]

As shown in FIG. 2B and FIG. 2C, the cap 50 is attached to the attaching device 70 and also attached to the distal end hard portion 21 by using the attaching device 70. In other words, the attaching device 70 attaches the cap 50 to the distal end hard portion 21 that functions as the inserting distal end portion of the inserting section 20 having the distal end surface 21a. In a case where the attaching device 70 attaches the cap 50 to the distal end hard portion 21, a central axis of the cap 50, a central axis of the distal end hard portion 21 and a central axis of the attaching device 70 are disposed coaxially with one another. As described above, the distal end hard portion 21 has the distal end surface 21a in which the functional region portion 21b including the opening portion such as the treatment instrument opening portion 130a and the non-functional region portion 21c that is a region excluding the functional region portion 21b are disposed. The attaching device 70 is made of, for example, a resin, a metal or the like.

As shown in FIG. 2B and FIG. 2C, the attaching device 70 has a distal end portion (hereinafter referred to as an attaching distal end portion 70a) and a proximal end portion (hereinafter referred to as an attaching proximal end portion 70b). The attaching device 70 is formed in a stepped shape so that the attaching distal end portion 70a of the attaching device 70 is thinner than the attaching proximal end portion 70b of the attaching device 70. Consequently, the attaching device 70 has, for example, a convex outer shape. The attaching distal end portion 70a is integral with the attaching proximal end portion 70b. The cap 50, the attaching device 70 and the distal end hard portion 21 are each separate members.

As shown in FIG. 2B and FIG. 2C, when the attaching device 70 is attached to the distal end hard portion 21, in an axial direction of the attaching device 70, the attaching distal end portion 70a is disposed close to the distal end hard portion 21 and the attaching proximal end portion 70b is disposed away from the distal end hard portion 21. When the cap 50 is attached to the attaching device 70, the attaching distal end portion 70a is covered with the cap 50 and the attaching proximal end portion 70b is exposed to the cap 50.

[Configuration 1 of Attaching Device 70]

As shown in FIG. 2B and FIG. 2C, the attaching device 70 has an attaching inserting section 71 that is disposed in the attaching distal end portion 70a and inserted to the opening portion such as the treatment instrument opening portion 130a when the attaching device 70 attaches the cap 50 to the distal end hard portion 21, and an abutment section 73 that is disposed in the attaching distal end portion 70a and abuts on at least a part of the non-functional region portion 21c when the attaching inserting section 71 is inserted to the opening portion. The attaching device 70 further has a base section 75 that is disposed in the attaching distal end portion 70a and on which the abutment section 73 is mounted. In this way, the attaching device 70 has the attaching inserting section 71, the abutment section 73 and the base section 75 in order from the distal end hard portion 21 in the axial direction of the attaching device 70.

The attaching inserting section 71, the abutment section 73 and the base section 75 constitute an integral member. In the axial direction of the attaching device 70, the attaching inserting section 71 is connected to the abutment section 73 and the abutment section 73 is connected to the base section 75.

[Attaching Inserting Section 71]

As shown in FIG. 2B and FIG. 2C, the attaching inserting section 71 is insertable into and removable from, for example, the treatment instrument opening portion 130a so that the attaching device 70 is attachable to and detachable from the distal end hard portion 21. The attaching inserting section 71 has, for example, a pillar shape, and specifically has a columnar shape or a polygonal pillar shape. An outer shape of the attaching inserting section 71 is about the same as an inner shape of the treatment instrument opening portion 130a. An outer diameter of the attaching inserting section 71 is about the same as an inner diameter of the treatment instrument opening portion 130a.

As shown in FIG. 2B, FIG. 2C and FIG. 2D, the attaching inserting section 71 is disposed at a position different from that on the central axis of the attaching device 70, so that the attaching inserting section 71 is inserted to the treatment instrument opening portion 130a. For example, the attaching inserting section 71 is disposed eccentrically from the central axis of the attaching device 70 toward an edge portion side of the attaching device 70. That is, the attaching inserting section 71 is eccentric to the central axis of the attaching device 70. In detail, the attaching inserting section 71 has a central axis eccentric to the central axis of the attaching device 70. The attaching inserting section 71 is disposed on an edge portion side of an abutment distal end surface portion 73c so that the attaching inserting section 71 is eccentric to the central axis of the attaching device 70 and a central axis of the abutment section 73. Details will be described later, but the attaching inserting section 71 is vertically disposed on the abutment section 73 so that the attaching inserting section 71 is disposed along an orthogonal direction orthogonal to a planar direction of the abutment distal end surface portion 73c and is further disposed along the axial direction of the attaching device 70. The attaching inserting section 71 is disposed in accordance with a positional relation among the central axis of the attaching device 70 and the central axis of the distal end hard portion 21 and the treatment instrument opening portion 130a.

[Abutment Section 73]

As shown in FIG. 2B and FIG. 2C, the abutment section 73 has, for example, a pillar shape, and specifically has a substantially ¼ columnar shape. The abutment section 73 is thicker than, for example, the attaching inserting section 71. The abutment section 73 is longer than, for example, a projecting length of the gas sending/water sending nozzle 120a.

As shown in FIG. 2B, FIG. 2C and FIG. 2D, the abutment section 73 is disposed so that the central axis of the abutment section 73 shifts from the central axis of the attaching device 70 in the radial direction of the attaching device 70. That is, the abutment section 73 is eccentric to the central axis of the attaching device 70.

As shown in FIG. 2B, FIG. 2C and FIG. 2D, the abutment section 73 has the abutment distal end surface portion 73c that is disposed in an abutment distal end portion 73a of the abutment section 73 and abuts on at least a part of the non-functional region portion 21c except the functional region portion 21b. The abutment distal end surface portion 73c is formed in, for example, a planar shape. In the present embodiment, the non-functional region portion 21c on which the abutment distal end surface portion 73c abuts indicates, for example, a region portion between the treatment instrument opening portion 130a and each observation window portion 100a in the periaxial direction of the distal end hard portion 21. The abutment section 73 including the abutment distal end surface portion 73c abuts only on the non-functional region portion 21c, but does not abut on the functional region portion 21b.

As shown in FIG. 2B, FIG. 2C and FIG. 2D, the abutment distal end surface portion 73c is connected to, for example, an attaching inserting proximal end portion 71b of the attaching inserting section 71 which is a root portion of the attaching inserting section 71. When the attaching inserting section 71 is inserted to the treatment instrument opening portion 130a, the abutment distal end surface portion 73c abuts on the non-functional region portion 21c mentioned above, and hence the abutment distal end surface portion 73c is connected to the attaching inserting proximal end portion 71b so that the attaching inserting section 71 is mounted on the abutment distal end surface portion 73c in the axial direction of the attaching device 70. In detail, the attaching inserting section 71 is disposed on the edge portion side of the abutment distal end surface portion 73c so that the central axis of the attaching inserting section 71 is not disposed on a central axis of the abutment distal end surface portion 73c and the central axis of the attaching device 70, so that the central axis of the attaching inserting section 71 shifts from the central axis of the abutment distal end surface portion 73c and the central axis of the attaching device 70 in the planar direction of the abutment distal end surface portion 73c, and so that the central axis of the attaching inserting section 71 is offset to the central axis of the abutment distal end surface portion 73c and the central axis of the attaching device 70.

[Base Section 75]

As shown in FIG. 2B and FIG. 2C, the base section 75 has, for example, a pillar shape, and specifically has a columnar shape. The base section 75 is thicker than, for example, the abutment section 73. A central axis of the base section 75 is disposed on the central axis of the attaching device 70.

As shown in FIG. 2B, FIG. 2C and FIG. 2D, the base section 75 has a base distal end surface portion 75c disposed in a base distal end portion 75a of the base section 75. The abutment section 73 is disposed in the base distal end surface portion 75c. The base distal end surface portion 75c is disposed away from the abutment distal end surface portion 73c of the abutment section 73 in an axial direction of the cap 50. The base distal end surface portion 75c is connected to an abutment proximal end portion 73b of the abutment section 73 which is a root portion of the abutment section 73 so that the abutment distal end surface portion 73c abuts on the non-functional region portion 21c mentioned above. The base distal end surface portion 75c is connected to the abutment proximal end portion 73b so that the abutment section 73 is mounted on the base distal end surface portion 75c in the axial direction of the attaching device 70. In detail, the abutment section 73 is disposed on an edge portion side of the base section 75 so that the central axis of the abutment section 73 is not disposed on a central axis of the base distal end surface portion 75c, so that the central axis of the abutment section 73 shifts from the central axis of the base distal end surface portion 75c in a planar direction of the base distal end surface portion 75c and so that the central axis of the abutment section 73 is offset to the central axis of the base distal end surface portion 75c.

As shown in FIG. 2C, the base section 75 is disposed away from the distal end surface 21a as much as a length of the abutment section 73 so that a space portion 80 is formed between the whole base distal end surface portion 75c of the base section 75 and the distal end surface 21a in the axial direction of the cap 50, when the abutment section 73 abuts on the non-functional region portion 21c. That is, the base section 75 does not abut on the distal end surface 21a.

As shown in FIG. 2B, FIG. 2C and FIG. 2D, the distal end portion 53 of the cap 50 is detachably attached to the base section 75. Further, an outer shape of the base section 75 is about the same as an inner shape of the cap 50 and an outer diameter of the base section 75 is about the same as an inner diameter of the cap 50 so that the distal end portion 53 of the cap 50 is fitted and attached into the base section 75. Consequently, an outer peripheral surface of the base section 75 is contactable closely with an inner peripheral surface of the cap 50. When the distal end portion 53 of the cap 50 is fitted and attached into the base section 75, the base section 75 positions the cap 50 to the attaching device 70 in the radial direction of the cap 50.

[Configuration 2 of Attaching Device 70]

[Positioning Section 77]

As shown in FIG. 2B, FIG. 2C and FIG. 2D, the attaching device 70 further has a positioning section 77 that positions the cap 50 and the attaching device 70 to the distal end hard portion 21 in a periaxial direction of the inserting section 20 by abutment of the distal end surface 53c of the cap 50 on the positioning section 77, thereby preventing the cap 50 and the attaching device 70 from rotating to the distal end hard portion 21 in the periaxial direction, when the attaching inserting section 71 is inserted into the opening portion such as the treatment instrument opening portion 130a, the abutment distal end surface portion 73c of the abutment section 73 abuts on at least a part of the non-functional region portion 21c and the attaching device 70 attaches the cap 50 to the distal end hard portion 21. The positioning section 77 being disposed around the base section 75.

As described above, the cap 50 has the concave portions 53a and the convex portions 53b disposed in the distal end surface 53c. The concave portions 53a and the convex portions 53b are alternately arranged in the periaxial direction of the cap 50. The distal end hard portion 21 has the observation unit and the illumination unit.

The positioning section 77 positions the cap 50 and the attaching device 70 at desirable positions to the distal end hard portion 21 in the periaxial direction in a state where, for example, the concave portion 53a is disposed adjacent to the observation window portion 100a of the observation unit or the illumination window portion 110a of the illumination unit in the radial direction of the cap 50.

As shown in FIG. 2B, FIG. 2C and FIG. 2D, the positioning section 77 therefore has convex portions 77a to be fitted into the concave portions 53a and concave portions 77b into which the convex portions 53b are to be fitted.

The convex portions 77a and the concave portions 77b are alternately arranged in a periaxial direction of the attaching device 70.

When the convex portions 77a are fitted into the concave portions 53a and the convex portions 53b are fitted into the concave portions 77b, the convex portions 77a and the concave portions 77b, together with the concave portions 53a and the convex portions 53b, position the cap 50 and the attaching device 70 to the distal end hard portion 21 in the periaxial direction. Further, the positioning section 77 positions the cap 50 to the attaching device 70 in the periaxial direction of the cap 50.

In detail, when the cap 50 is attached to the distal end hard portion 21, the concave portions 53a need to overlap with the observation view field 101 and the illumination range as described above. Consequently, the attaching device 70 needs to position the cap 50 to the distal end hard portion 21 in the periaxial direction of the distal end hard portion 21 so that the concave portions 53a overlap with the observation view field 101 and the illumination range.

For this purpose, first, the cap 50 is attached to the attaching device 70. At this time, the convex portions 77a are fitted into the concave portions 53a and the convex portions 53b are fitted into the concave portions 77b. Consequently, the cap 50 is positioned to the attaching device 70 in the periaxial direction of the attaching device 70.

Next, the attaching inserting section 71 is inserted to the treatment instrument opening portion 130a and the cap 50 is attached to the distal end hard portion 21. In consequence, the attaching device 70 is positioned to the distal end hard portion 21 in the periaxial direction of the distal end hard portion 21 by using the cap 50.

Further, the attaching device 70 positions the cap 50 to the distal end hard portion 21 in the periaxial direction of the distal end hard portion 21.

In detail, as shown in FIG. 2B, FIG. 2C and FIG. 2D, in the present embodiment, the treatment instrument opening portion 130a is eccentric to the central axis of the distal end surface 21a and the attaching inserting section 71 is eccentric to the central axis of the attaching device 70. Consequently, even when the attaching device 70 is to rotate in the periaxial direction of the attaching device 70 based on the attaching inserting section 71, the attaching device 70 is prevented from rotating in the periaxial direction of the attaching device 70 by the cap 50. In consequence, as described above, the attaching device 70 is positioned to the distal end hard portion 21 in the periaxial direction of the distal end hard portion 21 by using the cap 50. Additionally, as described above, the cap 50 is also prevented from rotating in the periaxial direction of the cap 50 by the attaching device 70.

As shown in FIG. 2B, FIG. 2C and FIG. 2D, as described above, a shape of the convex portions 77a corresponds to a shape of the concave portions 53a and a shape of the concave portions 77b corresponds to a shape of the convex portions 53b. Therefore, by the above positioning, the attaching device 70 attaches the cap 50 to the distal end hard portion 21 and also positions the cap 50 to the distal end hard portion 21 so that the concave portions 53a overlap with the observation view field 101 and the illumination range. In this way, the positioning section 77 may include the treatment instrument opening portion 130a, the attaching inserting section 71 and the cap 50.

Additionally, differently from the present embodiment, for example, the attaching inserting section 71 is disposed on the central axis of the attaching device 70 and the treatment instrument opening portion 130a is disposed on the central axis of the distal end surface 21a. In this case, when the attaching device 70 and the cap 50 are attached to the distal end hard portion 21, the attaching device 70 and the cap 50 are rotatable to the distal end hard portion 21 in the periaxial direction of the distal end hard portion 21. However, in the present embodiment, by the abovementioned eccentricity, this rotation is prevented.

It is to be noted that when there is no eccentricity, the rotation is performed. Then, by this rotation, the abutment section 73 slides on the distal end surface 21a in a state where the abutment section 73 abuts on the distal end surface 21a. This sliding includes moving from the non-functional region portion 21c to the functional region portion 21b and further moving in the functional region portion 21b. When the abutment section 73 moves in the functional region portion 21b in the state where the abutment section 73 abuts on the functional region portion 21b, the abutment section 73 might damage the functional region portion 21b due to friction. By this rotation, the concave portions 53a and the convex portions 53b might shift in the periaxial direction of the distal end hard portion 21, the convex portions 53b might overlap with the observation view field 101 and the illumination range, and the convex portions 53b might obstruct the observation view field 101 and the illumination range.

However, as shown in FIG. 2B, FIG. 2C and FIG. 2D, this rotation is prevented by the abovementioned eccentricity in the present embodiment. Therefore, the cap 50 is positioned to the distal end hard portion 21 in the periaxial direction of the distal end hard portion 21 by use of the attaching device 70 so that the concave portions 53a overlap with the observation view field 101 and the illumination range. The functional region portion 21b is prevented from being damaged by the abutment section 73 as described above. The concave portions 53a and the convex portions 53b are prevented from shifting in the periaxial direction of the distal end hard portion 21, the convex portions 53b are prevented from overlapping with the observation view field 101 and the illumination range, and the convex portions 53b are prevented from obstructing the observation view field 101 and the illumination range as described above.

Based on the above assumption, arrangement positions of the concave portions 53a and the convex portions 53b and those of the convex portions 77a and the concave portions 77b corresponding to the concave portions 53a and the convex portions 53b are desirably defined. When the cap 50 attached to the attaching device 70 is attached to the distal end hard portion 21, the attaching inserting section 71 is disposed in the abutment section 73 to position the cap 50 in the periaxial direction of the distal end hard portion 21 in the state where the concave portions 53a overlap with the observation view field 101 and the illumination range.

[Main Body Section 79]

As shown in FIG. 2B, FIG. 2C and FIG. 2D, for the positioning section 77, the attaching device 70 further has a main body section 79 that is disposed in the attaching proximal end portion 70b and is thicker than the base section 75. In an operation in which the cap 50 is attached to the attaching device 70, the main body section 79 functions as a grasping section by which the operator grasps the attaching device 70.

The main body section 79 has, for example, a pillar shape, and specifically has a columnar shape. The main body section 79 is integral with, for example, the base section 75. A central axis of the main body section 79 is disposed on the central axis of the attaching device 70.

As shown in FIG. 2B, FIG. 2C and FIG. 2D, the main body section 79 has a main body distal end surface portion 79c disposed in a main body distal end portion 79a of the main body section 79. The main body distal end surface portion 79c is connected to a base proximal end portion 75b of the base section 75 which is a root portion of the base section 75 so that the abutment distal end surface portion 73c abuts on the non-functional region portion 21c. The main body distal end surface portion 79c is connected to the base proximal end portion 75b of the base section 75 so that the base section 75 is mounted on the main body distal end surface portion 79c in the axial direction of the attaching device 70. In detail, the base section 75 is disposed in the main body section 79 so that the central axis of the base section 75 is disposed on a central axis of the main body distal end surface portion 79c. Consequently, the main body section 79 has the main body distal end surface portion 79c on which the base section 75 is mounted.

As described above, the main body section 79 is thicker than the base section 75 and the central axis of the main body section 79 is disposed coaxially with the central axis of the base section 75. According to this configuration, the main body distal end surface portion 79c has a ring-shaped exposed surface 79d disposed on a flat surface exposed from the base section 75.

As shown in FIG. 2B, FIG. 2C and FIG. 2D, the exposed surface 79d abuts on the distal end surface 53c of the cap 50, when the cap 50 is attached to the attaching device 70. Therefore, the exposed surface 79d has, for example, a shape corresponding to a shape of the distal end surface 53c of the cap 50 which abuts on the exposed surface 79d. In detail, the exposed surface 79d has the convex portion 77a in which a part of the exposed surface 79d is made convex as described above and the concave portion 77b in which a part of the exposed surface 79d is made concave as described above in the axial direction of the attaching device 70. As described above, in the periaxial direction of the attaching device 70, the convex portions 77a and the concave portions 77b are adjacent to one another and alternately arranged. The convex portions 77a are integral with a side surface of the base section 75.

The exposed surface 79d having the convex portions 77a and the concave portions 77b in this way are formed in, for example, a wave shape in the periaxial direction of the attaching device 70. In the periaxial direction of the attaching device 70, a ridgeline portion 79e of the exposed surface 79d is smoothened. That is, the convex portions 77a are smoothly continuous with the concave portions 77b without forming any stepped portions in the periaxial direction of the attaching device 70.

Further, as shown in FIG. 2B, FIG. 2C and FIG. 2D, when the cap 50 is attached to the attaching device 70, the exposed surface 79d abuts on the distal end surface 53c of the cap 50 so that the convex portions 53b are fitted into the concave portions 77b and the convex portions 77a are fitted into the concave portions 53a. Further, the exposed surface 79d prevents the cap 50 from passing through the attaching device 70 in the axial direction of the attaching device 70, by its abutment. The exposed surface 79d positions the cap 50 to the attaching device 70 in the axial direction of the attaching device 70. The convex portions 77a and the concave portions 77b, together with the concave portions 53a and the convex portions 53b, position the cap 50 to the attaching device 70 in the periaxial direction of the cap 50. As described above, the convex portions 77a and the concave portions 77b, together with the concave portions 53a and the convex portions 53b, position the cap 50 and the attaching device 70 to the distal end hard portion 21 in the periaxial direction.

[Length Relation]

As shown in FIG. 3A and FIG. 3E, as one example, lengths of respective members are defined as follows.

A length of the attaching inserting section 71 is referred to as L1.

A length of the attaching distal end portion 70a, i.e., a length from the exposed surface 79d to an attaching inserting distal end portion 71a of the attaching inserting section 71 is referred to as L2.

A total length of the cap 50 is referred to as L3.

The above lengths have a relation of L1<L3<L2.

Consequently, the attaching inserting section 71 has a length along which the attaching inserting distal end portion 71a of the attaching inserting section 71 passes through the cap 50 and projects from the proximal end portion 51 of the cap 50 to be exposed from the proximal end portion 51, when the distal end surface 53c abuts on the positioning section 77.

Consequently, when the cap 50 is attached to the attaching device 70, the attaching inserting distal end portion 71a is securely exposed from the proximal end portion 51 of the cap 50. In consequence, when the cap 50 including the attaching device 70 is attached to the distal end hard portion 21, the attaching distal end portion 70a is securely visually checked, and the attaching is easily performed.

As shown in FIG. 3A, a sum of a length of the abutment section 73 and a length of the base section 75 is referred to as L4. L4 is L2−L1.

This L4 defines the projecting amount of the cap 50 in the cap 50 projecting forward from the distal end surface 21a in the axial direction of the cap 50. Further, by using this L4, the concave portions 53a and the convex portions 53b to the distal end surface 21a are positioned in the axial direction of the cap 50 so that the concave portions 53a overlap with the observation view field 101 and the illumination range, when the cap 50 is attached to the distal end hard portion 21. That is, height positions of the concave portions 53a and the convex portions 53b to the distal end surface 21a are set by using the L4.

As shown in FIG. 3A and FIG. 3C, a treatment instrument inserting channel 130 has a cylindrical hard member 130b that is disposed in a distal end portion of the treatment instrument inserting channel 130 and communicates with the treatment instrument opening portion 130a, and a cylindrical soft member 130c that is disposed in a proximal end portion of the treatment instrument inserting channel 130 and communicates with the hard member 130b. The hard member 130b is, for example, a metal and the soft member 130c is, for example, a resin. A total length of the hard member 130b is referred to as L5.

The above lengths have a relation of L1<L5.

In this way, the attaching inserting section 71 is shorter than the hard member 130b in the axial direction of the cap 70 so that the attaching inserting section 71 is received only in the hard member 130b.

Consequently, the attaching inserting section 71 is received only in the hard member 130b, is not inserted into the soft member 130c, and prevents the soft member 130c from being damaged.

As shown in FIG. 3A and FIG. 3B, as one example, diameters of the respective members are defined as follows.

A diameter of the main body section 79 is referred to as D1.

An outer diameter of the cap 50 is referred to as D2.

The above diameters have a relation of D2≦D1.

In consequence, when the cap 50 is attached to the attaching device 70, the distal end surface 53c of the cap 50 securely abuts on the exposed surface 79d, and by this abutment, the exposed surface 79d prevents the cap 50 from passing through the attaching device 70 in the axial direction of the attaching device 70.

[Operation]

As shown in FIG. 2B and FIG. 3B, the distal end portion 53 of the cap 50 is fitted into the base section 75 so that the cap 50 covers the attaching distal end portion 70a. Further, the exposed surface 79d abuts on the distal end surface 53c of the cap 50 so that the convex portions 53b are fitted into the concave portions 77b and the convex portions 77a are fitted into the concave portions 53a. Consequently, the cap 50 is attached to the attaching device 70.

At this time, as shown in FIG. 2C, the cap 50 is prevented, by the exposed surface 79d, from passing through the attaching device 70 in the axial direction of the attaching device 70. Further, the cap 50 is positioned to the attaching device 70 in the axial direction of the attaching device 70 by use of the exposed surface 79d. The cap 50 is positioned to the attaching device 70 in the periaxial direction of the cap 50 by use of the convex portions 77a, the concave portions 77b, the concave portions 53a and the convex portions 53b. The cap 50 is positioned to the attaching device 70 in the radial direction of the cap 50 by use of the base section 75. As shown in FIG. 3B, the attaching inserting distal end portion 71a of the attaching inserting section 71 passes through the cap 50 and projects from the proximal end portion 51 of the cap 50 to be exposed from the proximal end portion 51.

Next, the main body section 79 is grasped. Further, as shown in FIG. 2B and FIG. 2C, the cap 50 is attached to the distal end hard portion 21 by using the attaching device 70 so that the attaching inserting section 71 is inserted to the treatment instrument opening portion 130a and the abutment section 73 abuts on the non-functional region portion 21c. As described above, the attaching inserting distal end portion 71a is exposed from the proximal end portion 51 of the cap 50, and hence the cap 50 is attached to the distal end hard portion 21 by using the attaching device 70 in a state where the attaching inserting distal end portion 71a is visually checked.

At this time, the attaching device 70 is to rotate to the distal end hard portion 21 in a periaxial direction of the attaching inserting section 71 based on the central axis of the attaching inserting section 71. However, the treatment instrument opening portion 130a is eccentric to the central axis of the distal end surface 21a, the attaching inserting section 71 is eccentric to the central axis of the attaching device 70, and there are disposed the convex portions 77a the concave portions 77b, the concave portions 53a and the convex portions 53b in the positioning section 77, and the cap 50. Therefore, the attaching device 70 is prevented from being rotated. Even when the cap 50 is to rotate to the distal end hard portion 21 in the periaxial direction of the cap 50, the cap 50 is prevented from being rotated as described above. Further, the cap 50 and the attaching device 70 are positioned to the distal end hard portion 21 in the periaxial direction of the distal end hard portion 21.

Therefore, the functional region portion 21b is prevented from being damaged by the rotation of the attaching device 70.

As described above, when the cap 50 attached to the attaching device 70 is attached to the distal end hard portion 21 in accordance with the arrangement positions of the concave portions 53a and the convex portions 53b and those of the convex portions 77a and the concave portions 77b corresponding to the concave portions 53a and the convex portions 53b, the concave portions 53a simultaneously overlap with the observation view field 101 and the illumination range. Consequently, when the attaching device 70 is removed, the cap 50 positioned in the periaxial direction of the distal end hard portion 21 is attached. That is, after the attaching device 70 is removed, the position of the cap 50 does not have to be adjusted in the periaxial direction of the distal end hard portion 21 so that the concave portions 53a overlap with the observation view field 101 and the illumination range. Therefore, the attaching operation does not take time and labor.

In the distal end surface 21a, the non-functional region portion 21c only abuts on the abutment section 73 and the functional region portion 21b does not abut on the attaching device 70. The base section 75 is disposed away from the distal end surface 21a as much as the length of the abutment section 73. Further, in the axial direction of the cap 50, the space portion 80 is formed between the whole base distal end surface portion 75c of the base section 75 and the distal end surface 21a.

Therefore, the attaching device 70 is prevented from damaging the functional region portion 21b.

In the cap 50 projecting forward from the distal end surface 21a in the axial direction of the cap 50, the projecting amount of the cap 50 is defined in accordance with the length of the abutment section 73 and the length of the base section 75 for the cap 50. That is, in the axial direction of the cap 50, the concave portions 53a and the convex portions 53b are positioned to the distal end surface 21a so that the concave portions 53a overlap with the observation view field 101 and the illumination range.

Consequently, when the attaching device 70 is removed, the cap 50 positioned in the axial direction of the distal end hard portion 21 is attached so that the concave portions 53a overlap with the observation view field 101 and the illumination range. That is, after the attaching device 70 is removed, the position of the cap 50 does not have to be adjusted in the axial direction of the distal end hard portion 21 so that the concave portions 53a overlap with the observation view field 101 and the illumination range. Therefore, the attaching operation does not take time and labor.

The cap 50 is made of a soft material such as a resin. Differently from the present embodiment, when the cap 50 is directly attached to the distal end hard portion 21, the shape of the cap 50 might break, and the cap 50 might not easily be attached to the distal end hard portion 21.

However, in the present embodiment, the attaching device 70 is utilized. Therefore, the cap 50 acquires a desirable strength by using the attaching device 70. Consequently, when the cap 50 is attached to the distal end hard portion 21, the shape of the cap 50 is prevented from being broken, and the cap 50 is easily attached to the distal end hard portion 21.

The abutment section 73 is longer than the gas sending/water sending nozzle 120a. Consequently, when the abutment section 73 abuts on the non-functional region portion 21c, the base section 75 is prevented from abutting on the gas sending/water sending nozzle 120a, and the base section 75 is prevented from pushing or breaking the gas sending/water sending nozzle 120a.

[Effect]

As described above, in the present embodiment, the treatment instrument opening portion 130a is eccentric to the central axis of the distal end surface 21a, the attaching inserting section 71 is eccentric to the central axis of the attaching device 70, and there are disposed the convex portions 77a, the concave portions 77b, the concave portions 53a and the convex portions 53b in the positioning section 77, and the cap 50. Therefore, in the present embodiment, when the cap 50 is attached to the distal end hard portion 21 by using the attaching device 70, the cap 50 including the attaching device 70 can be prevented from rotating in the periaxial direction of the cap 50. Consequently, in the present embodiment, the functional region portion 21b can be prevented from being damaged by the rotation.

In the present embodiment, as described above, when the cap 50 attached to the attaching device 70 is attached to the distal end hard portion 21, the concave portions 53a can simultaneously overlap with the observation view field 101 and the illumination range. Therefore, in the present embodiment, after the attaching device 70 is removed, the position of the cap 50 does not have to be adjusted in the periaxial direction of the distal end hard portion 21 so that the concave portions 53a overlap with the observation view field 101 and the illumination range. Therefore, the attaching operation does not take time and labor.

According to the present embodiment, in the distal end surface 21a, the non-functional region portion 21c abuts only on the abutment section 73 and the functional region portion 21b does not abut on the attaching device 70. The base section 75 is disposed away from the distal end surface 21a as much as the length of the abutment section 73. Further, in the axial direction of the cap 50, the space portion 80 is formed between the whole base distal end surface portion 75c of the base section 75 and the distal end surface 21a. Therefore, in the present embodiment, the attaching device 70 can be prevented from damaging the functional region portion 21b.

According to the present embodiment, in the cap 50 projecting forward from the distal end surface 21a in the axial direction of the cap 50, the projecting amount of the cap 50 can be defined in accordance with the length of the abutment section 73 and the length of the base section 75. Therefore, in the present embodiment, when the attaching device 70 is removed, the position of the cap 50 does not have to be adjusted in the axial direction of the distal end hard portion 21 so that the concave portions 53a overlap with the observation view field 101 and the illumination range after the attaching device 70 is removed. Therefore, the attaching operation does not take time and labor.

In the present embodiment, the cap 50 can easily be attached to the distal end hard portion 21 by using the attaching device 70.

In the present embodiment, the exposed surface 79d can prevent the cap 50 from passing through the attaching device 70 in the axial direction of the attaching device 70. In the present embodiment, the cap 50 can be positioned to the attaching device 70 in the axial direction of the attaching device 70 by using the exposed surface 79d. In the present embodiment, the cap 50 can be positioned to the attaching device 70 in the periaxial direction of the cap 50 by use of the convex portions 77a, the concave portions 77b, the concave portions 53a and the convex portions 53b. In the present embodiment, the cap 50 can be positioned to the attaching device 70 in the radial direction of the cap 50 by use of the base section 75.

In the present embodiment, the attaching inserting distal end portion 71a of the attaching inserting section 71 passes through the cap 50 and projects from the proximal end portion 51 of the cap 50 to be exposed from the proximal end portion 51. Therefore, in the present embodiment, the cap 50 can be attached to the distal end hard portion 21 by using the attaching device 70 in the state where the attaching inserting distal end portion 71a is visually checked.

In the present embodiment, the attaching inserting section 71 is shorter than the hard member 130b so that the attaching inserting section 71 is received only in the hard member 130b. Consequently, in the present embodiment, the attaching inserting section 71 can be received only in the hard member 130b and the soft member 130c can be prevented from being damaged.

The abutment section 73 is longer than the projecting length of the gas sending/water sending nozzle 120a. Consequently, when the abutment section 73 abuts on the non-functional region portion 21c, the base section 75 can be prevented from abutting on the gas sending/water sending nozzle 120a, and the base section 75 can be prevented from pushing or breaking the gas sending/water sending nozzle 120a.

In the present embodiment, as described above, there can be provided the cap 50 attached to the inserting distal end portion (the distal end hard portion 21) of the inserting section 20 by using the attaching device 70.

In the present embodiment, as described above, there can be provided the endoscope apparatus 5 including the cap 50 to be attached to the inserting distal end portion (the distal end hard portion 21) by using the attaching device 70. In other words, according to the present embodiment, there can be provided the endoscope apparatus 5 having the endoscope inserting section to which the cap 50 is attached as described above.

[Others]

It is to be noted that the attaching inserting section 71, the abutment section 73 and the base section 75 may be separate members, respectively. The base section 75 may be a separate member from the main body section 79.

The abutment distal end surface portion 73c may abut on the whole non-functional region portion 21c. In this case, for example, the abutment distal end surface portion 73c has the same shape as in the non-functional region portion 21c.

The positioning section 77 has the convex portions 77a, the concave portions 77b, the concave portions 53a and the convex portions 53b, but the present invention does not have to be limited to this embodiment. The positioning section 77 may further have at least one of a convex portion and a concave portion disposed in the non-functional region portion 21c and the other concave or convex portion to be disposed in the abutment distal end surface portion 73c and fitted into the one convex or concave portion.

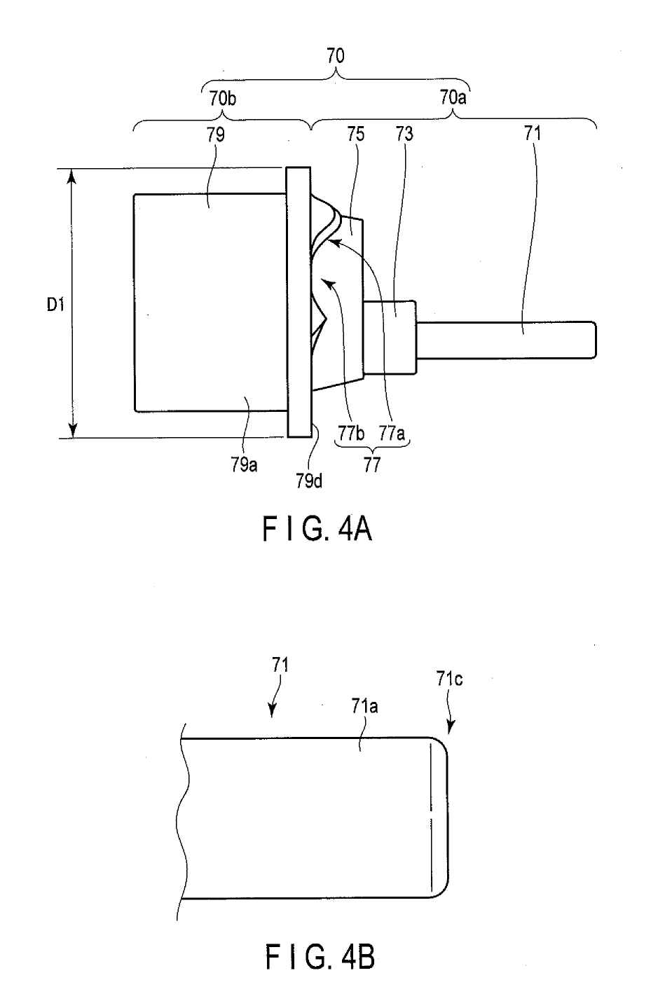

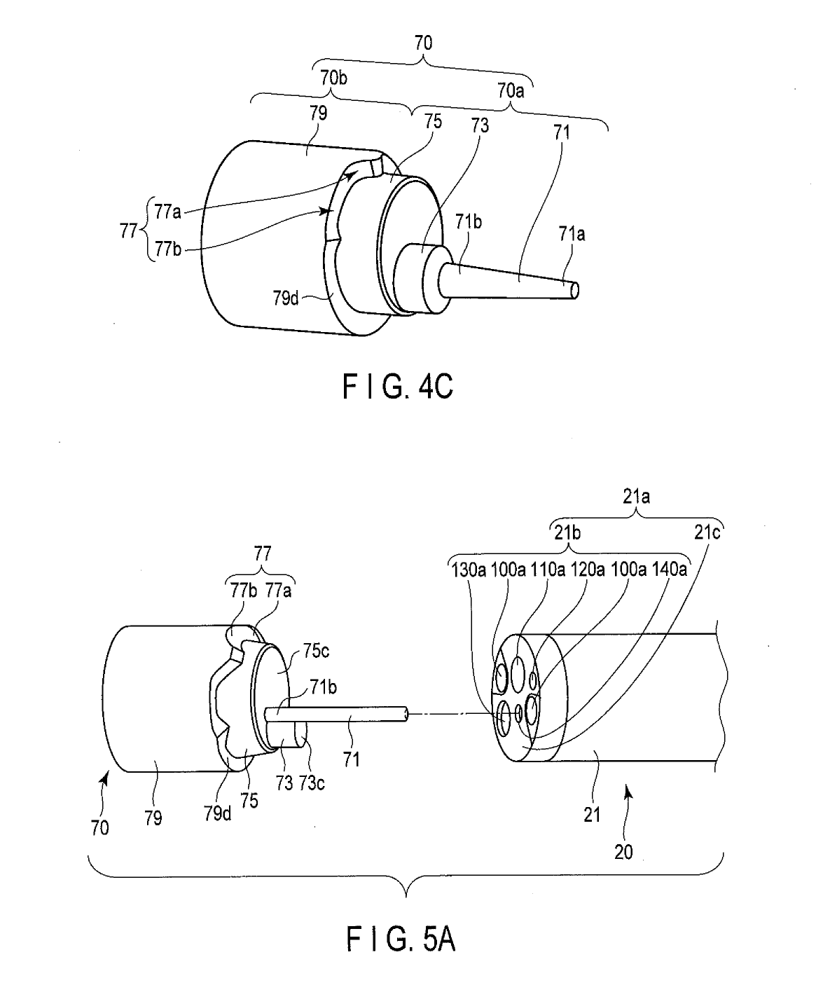

As shown in FIG. 4A, the main body distal end portion 79a of the main body section 79 including the exposed surface 79d may be formed as an outward flange portion. There is no special restriction on an outer shape of the ring-shaped exposed surface 79d, and the outer shape may be a circular or polygonal shape.

As shown in FIG. 4B, the attaching inserting distal end portion 71a of the attaching inserting section 71 may have a chamfered portion 71c obtained by chamfering the attaching inserting distal end portion 71a. Consequently, when the attaching inserting section 71 is inserted to the treatment instrument opening portion 130a, the attaching inserting distal end portion 71a can be prevented from damaging the treatment instrument opening portion 130a and the treatment instrument inserting channel 130, by use of the chamfered portion 71c.

As shown in FIG. 4C, the attaching inserting section 71 may be formed to be tapered from the attaching inserting proximal end portion 71b of the attaching inserting section 71 toward the attaching inserting distal end portion 71a of the attaching inserting section 71 in the axial direction of the attaching inserting section 71. Consequently, the attaching inserting section 71 can easily be inserted to the treatment instrument opening portion 130a.

The attaching inserting section 71 is inserted to the treatment instrument opening portion 130a, but the opening portion to which the attaching inserting section 71 is inserted is not limited to this treatment instrument opening portion.

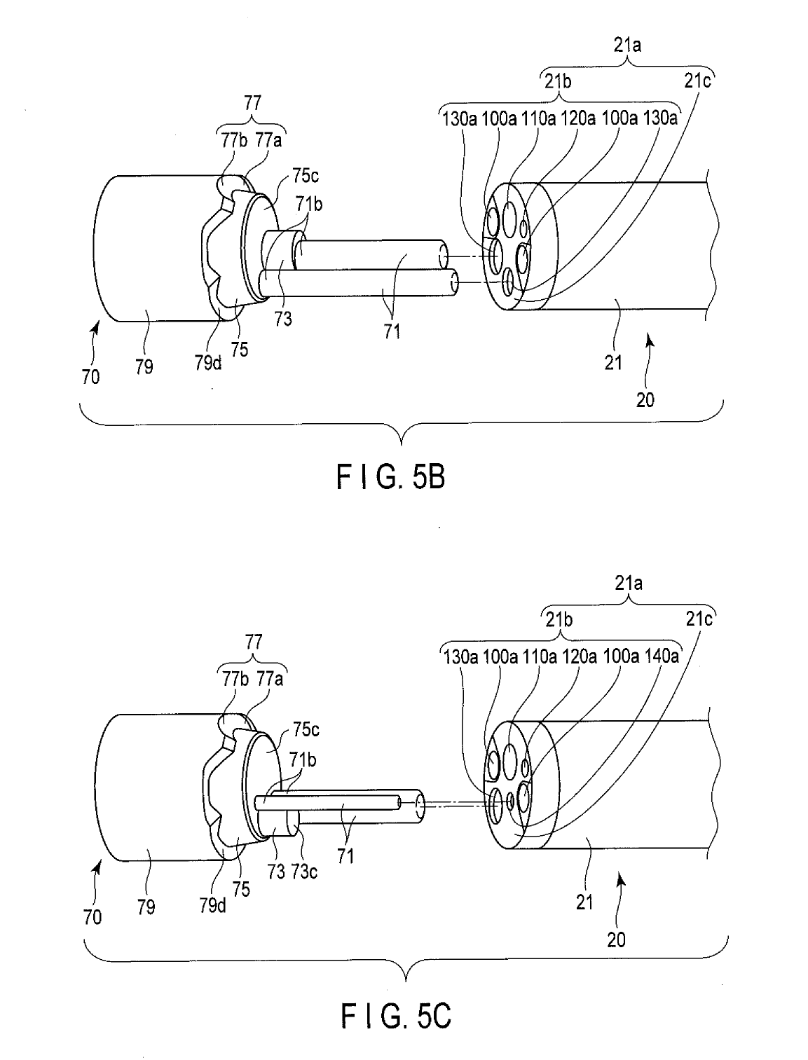

As shown in FIG. 5A, for example, the endoscope 10 has a discharge opening portion 140a that is disposed in the distal end surface 21a and discharges a washing liquid to wash the inside of a lumen, and a channel for the washing liquid which is disposed inside the inserting section 20 and communicates with the discharge opening portion 140a. In this case, as shown in FIG. 5A, the attaching inserting section 71 may be inserted to, for example, the discharge opening portion 140a. In this way, the opening portion to which the attaching inserting section 71 is inserted may be at least one of the treatment instrument opening portion 130a that communicates with the treatment instrument inserting channel 130 and the discharge opening portion 140a to discharge the washing liquid.

It is to be noted that similarly to the treatment instrument inserting channel 130 shown in FIG. 3C, the channel for the washing liquid has a cylindrical hard member that is disposed in a distal end portion of the channel for the washing liquid disposed inside the inserting section 20, and communicates with the discharge opening portion 140a, and a cylindrical soft member that is disposed in a proximal end portion of the channel for the washing liquid and communicates with the hard member. The hard member is, for example, a metal and the soft member 130c is, for example, a resin. The attaching inserting section 71 is shorter than the hard member.

In this way, the opening portion to which the attaching inserting section 71 is inserted communicates with the cylindrical hard member disposed in the distal end portion of the channel disposed inside the inserting section 20. Further, the attaching inserting section 71 is shorter than the hard member in the axial direction of the cap 70 so that the attaching inserting section is received only in the hard member.

As described above, the opening portions are disposed. In this case, as shown in FIG. 5B, for example, the treatment device opening portions 130a are disposed. Alternatively, as shown in FIG. 5A and FIG. 5C, the treatment instrument opening portion 130a and the discharge opening portion 140a are disposed. At least one or more attaching inserting sections 71 may be disposed, the same number of the attaching inserting sections 71 as the number of the opening portions may be disposed, or the number of the attaching inserting sections 71 which is smaller than the number of the opening portions may be disposed, and each of the attaching inserting sections 71 may be inserted to each of the opening portions.

The attaching inserting section 71 may be a separate member from the abutment section 73. In this case, as in the attaching inserting section 71 shown in FIG. 5A, FIG. 5B and FIG. 5C, the attaching inserting section 71 is connected to the base section 75 so that the attaching inserting proximal end portion 71b of the attaching inserting section 71 is connected to the base distal end surface portion 75c. That is, the attaching inserting section 71 may vertically be disposed on the base section 75 so that the attaching inserting section 71 is disposed along an orthogonal direction to a planar direction of the base distal end surface portion 75c and is further disposed along the axial direction of the attaching device 70. Further, the attaching inserting section 71 may vertically be disposed on at least one of the abutment section 73 and the base section 75.

In this case, as shown in FIG. 5B, one attaching inserting section 71 may be connected to the abutment section 73, and the other attaching inserting section 71 may be connected to the base section 75 so that the one attaching inserting section 71 is disposed in parallel with the other attaching inserting section 71. Alternatively, each attaching inserting section 71 may be connected to the abutment section 73, though not shown in the drawing, and the attaching inserting section 71 may be connected to the base section 75 as shown in FIG. 5C.

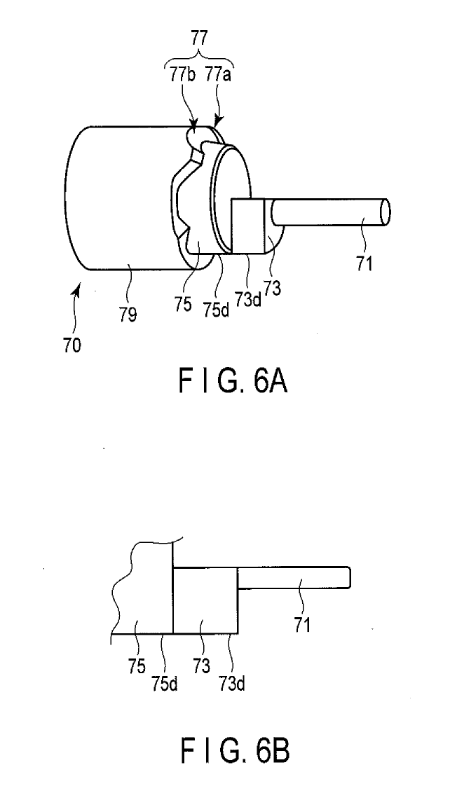

As shown in FIG. 6A and FIG. 6B, a part 73d of a circular outer peripheral surface of the abutment section 73 may be disposed on the same plane as in a part 75d of an outer peripheral surface of the base section 75 and connected to the part 75d of the outer peripheral surface of the base section 75 in the axial direction of the attaching device 70 so that the part 73d abuts on a part of an inner peripheral surface of the cap 50, when the cap 50 is attached to the attaching device 70. Consequently, the part 73d of the circular outer peripheral surface of the abutment section 73 can abut on a part of the inner peripheral surface of the cap 50. In consequence, when the cap 50 is attached to the attaching device 70, looseness of the cap 50 in the radial direction of the cap 50 can be prevented.

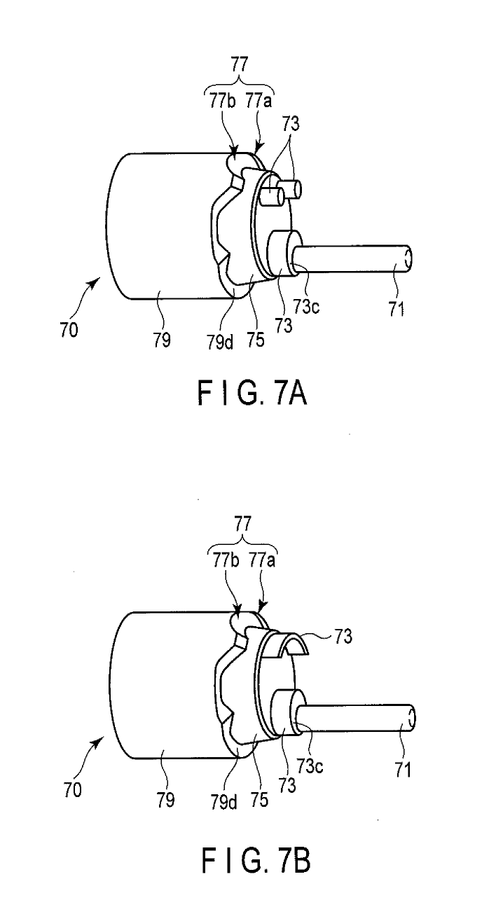

As shown in FIG. 7A and FIG. 7B, the abutment sections 73 may be disposed. In this case, the respective abutment sections 73 have the same length.

In this case, one abutment section 73 is connected to the attaching inserting proximal end portion 71b of the attaching inserting section 71 as described above. Further, as shown in FIG. 7A and FIG. 7B, the one abutment section 73 abuts on, for example, the non-functional region portion 21c indicating a region portion between the treatment instrument opening portion 130a and the observation window portion 100a in the periaxial direction of the distal end hard portion 21 as described above.

As shown in FIG. 7A and FIG. 7B, the other abutment section 73 does not abut on a side of the one abutment section 73, e.g., the gas sending/water sending nozzle 120a, but abuts on the non-functional region portion 21c that is a region portion around the observation window portion 100a. The other abutment section 73 is suitably disposed in an edge portion of the base distal end surface portion 75c. The other abutment section 73 may have a columnar shape as shown in FIG. 7A or may have a circular shape as shown in FIG. 7B.

The abutment sections 73 are disposed and all the abutment sections 73 abut on the non-functional region portion 21c, and hence looseness of the attaching device 70 including the abutment sections 73 can be suppressed and the attaching device 70 can securely be prevented from damaging the distal end surface 21a.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. An endoscope distal end cap attaching device which attaches an endoscope distal end cap to an inserting distal end portion of an endoscope inserting section having a distal end surface including a functional region portion in which a window portion for observation or illumination of a subject is disposed, one or more opening portions, and a non-functional region portion that is a region excluding the functional region portion and the opening portion,
the endoscope distal end cap attaching device comprising:
an attaching inserting section that is inserted to the opening portion, when the endoscope distal end cap attaching device attaches the endoscope distal end cap to the inserting distal end portion;
an abutment section having one or more abutment distal end surface portions that abut only on at least a part of the non-functional region portion except the functional region portion, when the attaching inserting section is inserted to the opening portion;
a base section which has a base distal end surface portion on which the abutment section is disposed, the base distal end surface portion being disposed away from the abutment distal end surface portion of the abutment section in an axial direction of the endoscope distal end cap; and
a positioning section which positions the endoscope distal end cap and the endoscope distal end cap attaching device in a periaxial direction of the endoscope inserting section by the abutment of a cap distal end surface of the endoscope distal end cap, when the abutment distal end surface portion abuts on the non-functional region portion, the positioning section being disposed around the base section.

2. The endoscope distal end cap attaching device according to claim 1,
wherein the cap distal end surface of the endoscope distal end cap has cap concave portions and cap convex portions alternately arranged in a periaxial direction of the endoscope distal end cap, and
the positioning section positions the endoscope distal end cap and the endoscope distal end cap attaching device to the inserting distal end portion in the periaxial direction so that the cap concave portion and the observation window portion of an observation unit or the illumination window portion of an illumination unit are arranged adjacent to each other in a radial direction of the endoscope distal end cap.

3. The endoscope distal end cap attaching device according to claim 2,
wherein in the positioning section, attaching convex portions to be fitted into the cap concave portions and attaching concave portions into which the cap convex portions are to be fitted are alternately arranged in the periaxial direction, and
the attaching concave portions into which the cap convex portions are fitted and the attaching convex portions fitted into the cap concave portions, together with the cap concave portions and the cap convex portions, position the endoscope distal end cap and the endoscope distal end cap attaching device to the inserting distal end portion in the periaxial direction.

4. The endoscope distal end cap attaching device according to claim 3, further comprising:
a main body section that has a main body distal end surface portion in which the base section is disposed, and is thicker than the base section,
wherein the main body distal end surface portion has the attaching convex portions and the attaching concave portions.

5. The endoscope distal end cap attaching device according to claim 1,
wherein the functional region portion further includes a gas sending/water sending nozzle that sends a gas and water toward the window portion to observe the subject.

6. The endoscope distal end cap attaching device according to claim 1,
wherein the attaching inserting section is formed to be tapered from a proximal end portion of the attaching inserting section toward a distal end portion of the attaching inserting section in an axial direction of the attaching inserting section.

7. The endoscope distal end cap attaching device according to claim 1,
wherein the opening portions have at least one of a treatment instrument opening portion that communicates with a treatment instrument inserting channel and a discharge opening portion to discharge a washing liquid.

8. The endoscope distal end cap attaching device according to claim 1,
wherein the opening portions are disposed, and
at least one or more attaching inserting sections are disposed, the same number of the attaching inserting sections as the number of the opening portions are disposed, or the number of the attaching inserting sections which is smaller than the number of the opening portions are disposed, and each of the attaching inserting sections is inserted to each of the opening portions.

9. The endoscope distal end cap attaching device according to claim 1,
wherein at least one of the attaching inserting sections has a length along which a distal end portion of the attaching inserting section projects from a proximal end portion of the endoscope distal end cap, when the cap distal end surface abuts on the positioning section.

10. The endoscope distal end cap attaching device according to claim 1,
wherein the opening portion communicates with a cylindrical hard member disposed in the inserting distal end portion of the endoscope inserting section, and
the attaching inserting section is shorter than the hard member in the axial direction of the endoscope distal end cap so that the attaching inserting section is received only in the hard member.

11. The endoscope distal end cap attaching device according to claim 1,
wherein the attaching inserting section is vertically disposed on at least one of the abutment section and the base section.

12. The endoscope distal end cap attaching device according to claim 1,
wherein the abutment distal end surface portions are disposed.

13. An endoscope distal end cap which is attached to the endoscope distal end cap attaching device according to claim 1 and the inserting distal end portion of the endoscope inserting section.

14. An endoscope apparatus which comprises the endoscope distal end cap attaching device which attaches an endoscope distal end cap to an inserting distal end portion of an endoscope inserting section having a distal end surface including a functional region portion in which a window portion for observation or illumination of a subject is disposed, one or more opening portions, and a non-functional region portion that is a region excluding the functional region portion and the opening portion,
the endoscope distal end cap attaching device comprising:
an attaching inserting section that is inserted to the opening portion, when the endoscope distal end cap attaching device attaches the endoscope distal end cap to the inserting distal end portion;
an abutment section having one or more abutment distal end surface portions that abut only on at least a part of the non-functional region portion except the functional region portion, when the attaching inserting section is inserted to the opening portion;
a base section which has a base distal end surface portion on which the abutment section is disposed, the base distal end surface portion being disposed away from the abutment distal end surface portion of the abutment section in an axial direction of the endoscope distal end cap;
a positioning section which positions the endoscope distal end cap and the endoscope distal end cap attaching device in a periaxial direction of the endoscope inserting section by the abutment of a cap distal end surface of the endoscope distal end cap, when the abutment distal end surface portion abuts on the non-functional region portion, the positioning section being disposed around the base section; and
the endoscope distal end cap according to claim 13.
